(19) United States
(12) Patent Application Publication
(10) Pub. No.: US 2022/0047422 A1
(43) Pub. Date: Feb. 17, 2022

(54) METHODS AND SYSTEMS FOR LASER ASSISTED TECHNOLOGY FOR MINIMALLY-INVASIVE AB-INTERNO GLAUCOMA SURGERY

(71) Applicant: IOPTIMA LTD., Caesarea (IL)

(72) Inventors: Eran KAPLAN, Tel Aviv (IL); Ronen CASTRO, Tel Aviv (IL)

(21) Appl. No.: 17/413,191

(22) PCT Filed: Dec. 12, 2019
(86) PCT No.: PCT/IB2019/001336

§ 371 Date: Jun. 11, 2021

Related U.S. Application Data
(60) Provisional application No. 62/779,221, filed on Dec. 13, 2018.

(51) Int. Cl. A61F 9/008

(52) U.S. Cl. CPC A61F 9/00802; A61F 2009/00891; A61F 2009/00868

(57) ABSTRACT

Some embodiments of the present disclosure relate to a method and system where a fiber optic probe is obtained. In some embodiments, the fiber optic probe comprises a distal end. In some embodiments, the fiber optic probe is introduced between an outer surface of an eye and an anterior chamber of an eye. In some embodiments, the fiber optic probe is advanced into one or more portions of the eye. In some embodiments, a plurality of pulses of laser radiation are delivered through a laser and into the eye. In some embodiments, the laser is disposed at a distal end of the fiber optic probe. In some embodiments, ocular tissue of the eye is ablated with the plurality of pulses of laser radiation. In some embodiments, the ablating generates a drainage channel that extends from the anterior chamber of the eye to the subconjunctival space of the eye.

### FIELD OF THE DISCLOSURE

The field of the disclosure relates to devices for use in laser surgery. More specifically, the present disclosure relates to a method and laser apparatus for treating glaucoma.

### BACKGROUND

Glaucoma, the leading cause of irreversible blindness in the world, is a group of diseases affecting the optic nerve, frequently characterized by increased intraocular pressure (“IOP”). Patients suffering from glaucoma are typically initially managed with medical therapy. However, some patients are unable to tolerate medication or do not adhere to their drug regimens, and, consequently, may require surgical intervention.

Without proper drainage of the aqueous humor from the anterior chamber, an abnormally high fluid pressure results within the eye which is referred to as glaucoma. As pressure builds up, the pressure can “pinch” both the optic nerve and the blood vessels which nourish the retina. The result is usually a slow loss of peripheral vision, and eventually blindness.

Therefore, glaucoma is treated by reducing the IOP, through improving aqueous humor outflow and/or reducing aqueous production.

However, some surgical interventions for reducing IOP can cause issues. For example, incisions can cause trauma to the eye and cause scar tissue to form in the interior chamber. This can cause IOP to build up again and lead to relapse. Conversely, certain surgical procedures can cause incisions that are too large to heal properly, causing low IOP, which is known as hypotony.

There is therefore a need in the art for methods and systems for treatment of glaucoma in a minimally invasive manner to reduce the likelihood of complications.

### SUMMARY

The exemplary embodiments of the present disclosure relate to methods and systems for treatment of glaucoma.

In some embodiments, the method includes providing a fiber optic probe comprising a distal end; introducing the fiber optic probe between the outer surface of an eye and the anterior chamber; advancing the distal end of the fiber optic probe until it is adjacent to or in contact with the trabecular meshwork Schwalbe's line, between the scleral spur and the sclerocorneal junction, or any combination thereof; delivering a plurality of pulses of radiation from a laser through the distal end of the fiber optic probe; and ablating ocular tissue of the eye with the plurality of pulses of radiation, wherein the ablating of the ocular tissue of the eye with the plurality of pulses of radiation generates a drainage channel; and wherein the drainage channel extends from the anterior chamber of the eye to the sub-conjunctival space of the eye.

In some embodiments, the system includes a fiber optic probe and a laser, wherein the fiber optic probe comprises a distal end; and wherein the fiber optic probe is configured to: deliver a plurality of pulses of radiation from the distal end; and ablate ocular tissue to form a drainage channel; and wherein the drainage channel extends from the anterior chamber of the eye to the sub-conjunctival space of the eye.

In some embodiments, the ablation is thermal ablation that is performed using a thermal laser.

In some embodiments, the fiber optic probe is inserted into the eye through a corneal incision.

In some embodiments, the fiber optic probe is inserted into the eye by perforation of the fiber optic probe.

In some embodiments, the fiber optic probe is guided for placement in contact with or adjacent to the trabecular meshwork through microscopic observation.

In some embodiments, the microscopic observation is aided by an aiming beam, wherein the aiming beam coupled to the laser beam, and wherein the aiming beam is on the visible spectrum.

In some embodiments, the fiber optic probe is guided for placement in contact with or adjacent to the trabecular meshwork by a goniolens.

In some embodiments, the fiber optic probe is guided for placement adjacent to or in contact with the trabecular meshwork by coupling the fiber optic probe with an endoscope.

In some embodiments, the endoscope comprises a camera and a light.

In some embodiments, the laser is configured to deliver radiation having a tissue absorption coefficient of 10 cm⁻¹ or more.

In some embodiments, the laser is configured to deliver radiation having a tissue absorption depth ranging from 1 μm to 0.6 mm.

In some embodiments, the laser is configured to deliver radiation having a tissue absorption depth of below 0.6 mm.

In some embodiments, the laser is configured to deliver radiation having a tissue absorption coefficient ranging from 10 cm⁻¹ to 12,000 cm⁻¹.

In some embodiments the laser is configured to deliver radiation having a wavelength of less than 11 μm.

In some embodiments, the laser is configured to deliver radiation having a wavelength of less than 2 μm.

In some embodiments, the laser is configured to deliver radiation having a wavelength ranging from 1 nm to 11 μm.

In some embodiments, the laser is configured to deliver radiation having a wavelength ranging from 2 μm to 11 μm and a tissue absorption coefficient ranging from 100 to 12,000 cm⁻¹.

In some embodiments, the laser can have any wavelength if the tissue absorption coefficient is above 10 cm⁻¹ or the absorption depth is below 0.6 mm.

[27] In some embodiments the laser comprises one or more of: an Eerbium-Chromium doped Yttrium Scandium Gallium Garnet laser, a fiber laser, a quantum cascade laser, a Holmium doped Yttrium Scandium Gallium Garnet laser, or a fiber laser.

In some embodiments, the laser is a carbon dioxide laser.

In some embodiments, the laser is an erbium-doped yttrium aluminum garnet laser.

In some embodiments, the laser is an Erbium, Chromium doped Yttrium Scandium Gallium Garnet laser having a wavelength of 2790 μm.

In some embodiments, the laser is a fiber laser configured to emit radiation having a wavelength in the range of 2.8 μm to 3.5 μm.

In some embodiments, the carbon dioxide laser is configured to deliver radiation having a wavelength of 10.6 μm.

In some embodiments, the erbium-doped yttrium aluminum laser is configured to deliver radiation having a wavelength of 6 μm.

In some embodiments, the erbium-doped yttrium aluminum garnet laser is configured to deliver radiation having a wavelength of 2.94 μm.

In some embodiments, each pulse of the plurality of pulses of laser radiation has a duration ranging from 10 μs to 1 s.

In some embodiments, the fiber optic probe inserted into the eye is straight.

In some embodiments, the fiber optic probe inserted into the eye is bent with a bending radius of up to 40°.

In some embodiments, the fiber optic probe is a solid core fiber.

In some embodiments, the fiber optic probe is a hollow core waveguide.

In some embodiments, the fiber optic probe has an additional cover for thermal insulation.

In some embodiments, the fiber optic probe is connected to a handpiece.

In some embodiments, the hollow core waveguide comprises an optical window at an exit portion of the hollow core wave guide.

In some embodiments, the optical window is a diamond or zinc-selenium window.

In some embodiments, the fiber optic probe comprises an inner annulus and an outer annulus, the method further comprising the steps of: emitting a fluid from the inner annulus of the fiber optic probe thereby irrigating the eye, the fluid having a temperature T₁; and aspirating fluid from the eye into the outer annulus of the fiber optic probe the air having a temperature T₂; wherein T₂>T₁, such that the receipt of fluid into the outer annulus cools the eye.

In some embodiments, the fluid comprises air.

In some embodiments, the inner annulus also transmits the pulses of laser radiation such that the medium for the laser is air.

In some embodiments, the method further comprises a step of injecting a viscoelastic material into the anterior chamber.

In some embodiments, the method further comprises a step of providing an anterior chamber maintainer.

In some embodiments, the method further comprises a step of injecting a liquid or viscoelastic material into the subconjunctival space.

In some embodiments, the liquid material comprises an anti-fibrotic material.

In some embodiments, the anti-fibrotic material comprises mitomycin-C.

In some embodiments, the anti-fibrotic material comprises fluorouracil

In some embodiments, the method further comprises a step of injecting viscoelastic material into the anterior chamber.

### DRAWINGS

Some embodiments of the disclosure are herein described, by way of example only, with reference to the accompanying drawings. With specific reference now to the drawings in detail, it is stressed that the embodiments shown are by way of example and for purposes of illustrative discussion of embodiments of the disclosure. In this regard, the description taken with the drawings makes apparent to those skilled in the art how embodiments of the disclosure may be practiced.

FIG. 1 shows an exemplary drainage channel created by embodiments of the methods and systems of the present disclosure.

FIG. 2 depicts the wavelengths and absorption coefficients corresponding to exemplary chromophores targeted by embodiments of methods and systems in accordance with the present disclosure.

FIG. 3 depicts the wavelengths and absorption coefficients corresponding to exemplary lasers used in embodiments of methods and systems in accordance with the present disclosure.

FIG. 4 is a cross-sectional view of a fiber optic probe according to some embodiments of the present disclosure.

FIG. 5 depicts several views of a fiber optic probe according to some embodiments of the present disclosure.

### DETAILED DESCRIPTION

Among those benefits and improvements that have been disclosed, other objects and advantages of this disclosure will become apparent from the following description taken in conjunction with the accompanying figures. Detailed embodiments of the present disclosure are disclosed herein; however, it is to be understood that the disclosed embodiments are merely illustrative of the disclosure that may be embodied in various forms. In addition, each of the examples given regarding the various embodiments of the disclosure which are intended to be illustrative, and not restrictive.

Throughout the specification and claims, the following terms take the meanings explicitly associated herein, unless the context clearly dictates otherwise. The phrases “in one embodiment,” “in an embodiment,” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments of the disclosure may be readily combined, without departing from the scope or spirit of the disclosure.

As used herein, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

The exemplary embodiments relate to a method and system for treating glaucoma. The method and system of the exemplary embodiments utilize an “ab interno” approach, in which a drainage channel is created from inside of the eye toward the outside. The “ab interno” approach is contrasted with the “ab externo” approach where the channel is created from the outside of the eye inward. In some embodiments, the “ab interno” approach entails advancing a device through the peripheral cornea and across the anterior chamber.

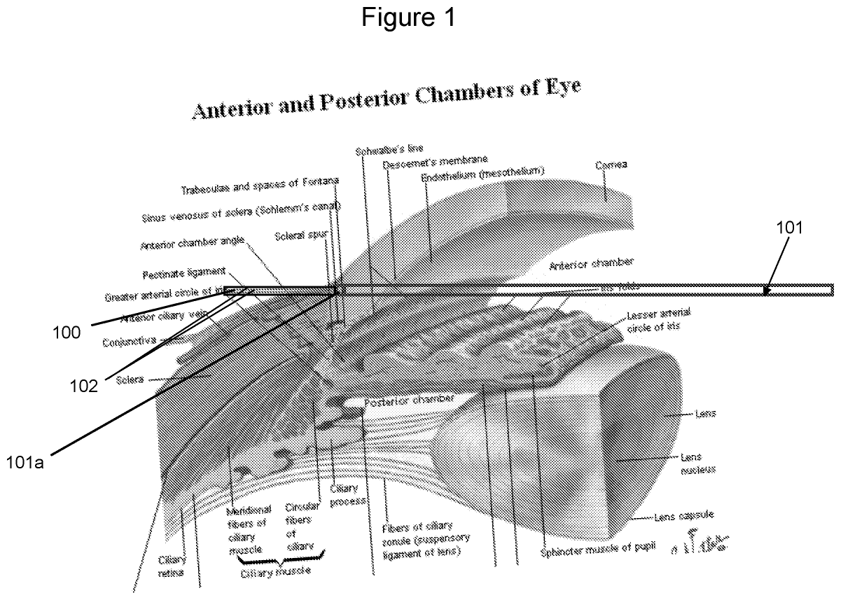

In an embodiment shown in FIG. 1, the method and system can entail the creation of a drainage channel 100. The drainage channel 100 can be formed by providing a fiber optic probe 101 comprising a distal end 101a and introducing the fiber optic probe 101 between an outer surface of an eye, such as the cornea, and the anterior chamber of the eye, until the distal end 101a of the fiber optic probe 101 is adjacent to or in contact with the trabecular meshwork.

As used herein, the term “adjacent to” means that the fiber optic probe 101 is a not in contact with the target tissue of the entrance point (i.e. trabecular meshwork, Schwalbe's line, or any point in the range between the scleral spur and the sclerocorneal junction) but is at a sufficient distance from the entrance point (i.e. trabecular meshwork, Schwalbe's line, or any point in the range between the scleral spur and the sclerocorneal junction) to deliver pulses of radiation thereto. Such a distance is not limited and can be determined by one of ordinary skill in the art. In some embodiments, this distance can range from 0-10 mm and all ranges there between. In some embodiments, this distance can be on the order of microns and can range from 0-100 μm, including all ranges there between.

In some embodiments, once the fiber optic probe 101 is adjacent to or in contact with the target tissue of the entrance point (i.e. trabecular meshwork, Schwalbe's line, or any point in the range between the scleral spur and the sclerocorneal junction), a plurality of pulses 102 of laser radiation can be emitted through the distal end 101a of the fiber optic probe 101. In some embodiments, the emission of the plurality of pulses 102 of laser radiation ablates the ocular tissue and generate the drainage channel 100, so that the drainage channel 100 extends from the anterior chamber of the eye to a sub-conjunctival space of the eye. In some embodiments, the laser is a thermal laser, such that the emission of the plurality of pulses 102 of laser radiation thermally ablates the ocular tissue to generate the drainage channel 100, so that the drainage channel 100 extends from the anterior chamber of the eye to a sub-conjunctival space of the eye.

As used herein, the term “subconjunctival space” is the area above the sclera and below the conjunctiva.

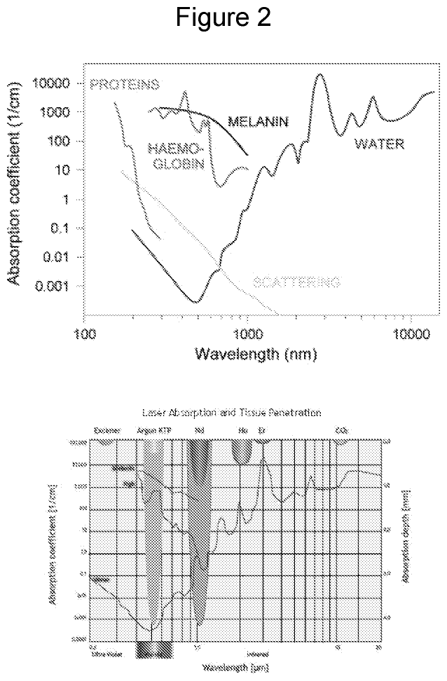

In some embodiments, the laser is a thermal laser and is selected to correspond to the target wavelength and absorption coefficient of water absorbing chromophores. As shown in FIG. 2, this can correspond to a wavelength in the infrared spectrum. For example, in some embodiments, the wavelength can range from 10 μm to 1,000 μm. In some embodiments, the wavelength can range from 10 μm to 100 μm. In some embodiments, the wavelength can range from 100 μm to 1,000 μm.

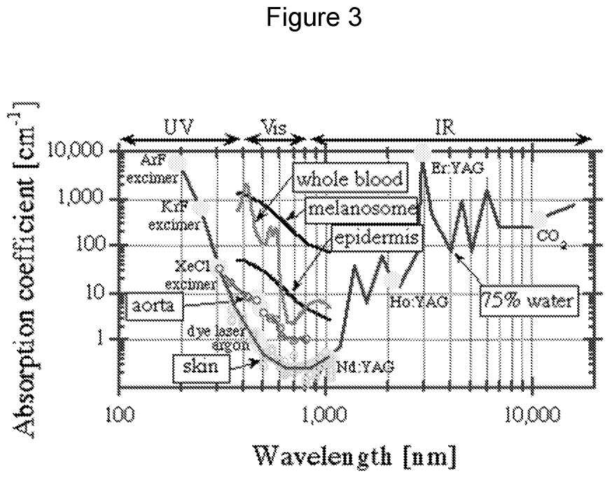

Exemplary thermal lasers for the creation of the drainage channel 100 are depicted in FIG. 3. As shown, exemplary having suitable wavelengths and absorption coefficients can include Carbon Dioxide (“CO₂”) lasers and erbium-doped yttrium aluminum garnet (“Er: YAG”) lasers. However, other suitable lasers having wavelengths and absorption coefficients corresponding to that of water can be used.

In some embodiments, lasers having wavelengths and absorption coefficients corresponding to water are used or configured to target the aqueous humor of the eye.

In some embodiments, the CO₂ lasers can deliver pulses 102 of radiation having a wavelength of 10.6 μm.

In some embodiments, the laser can include one or more of a: Erbium, Chromium doped Yttrium Scandium Gallium Garnet laser (“Er, Cr: YSGG”), a fiber laser, a quantum cascade laser, or a Holmium doped Yttrium Scandium Gallium Garnet laser (“Ho: YAG”), such as a Holmium doped Yttrium Scandium Gallium Garnet laser having an optical parametric oscillator (“Ho: YAG & OPO”).

In some embodiments, the laser is a Er: YAG laser configured to deliver pulses 102 of thermal radiation having a wavelength of 2.94 μm. In some embodiments, the Er: YAG laser is configured to deliver pulses 102 of radiation having a wavelength of 6 μm.

In some embodiments, the laser is an Er, Cr: YSGG laser having a wavelength of 2.790 μm.

In some embodiments, the laser is a thermal laser. In some embodiments, the thermal laser is a fiber laser configured to emit radiation having a wavelength in the range of 2.8 μm to 3.5 μm. In some embodiments, the thermal laser is a fiber laser configured to emit radiation having a wavelength in the range of 2.9 μm to 3.5 μm. In some embodiments, the thermal laser is a fiber laser configured to emit radiation having a wavelength in the range of 3.0 μm to 3.5 μm. In some embodiments, the thermal laser is a fiber laser configured to emit radiation having a wavelength in the range of 3.1 μm to 3.5 μm. In some embodiments, the thermal laser is a fiber laser configured to emit radiation having a wavelength in the range of 3.2 μm to 3.5 μm. In some embodiments, the thermal laser is a fiber laser configured to emit radiation having a wavelength in the range of 3.3 μm to 3.5 μm. In some embodiments, the thermal laser is a fiber laser configured to emit radiation having a wavelength in the range of 3.4 μm to 3.5 μm.

In some embodiments, the thermal laser is a fiber laser configured to emit radiation having a wavelength in the range of 2.8 μm to 3.4 μm. In some embodiments, the thermal laser is a fiber laser configured to emit radiation having a wavelength in the range of 2.8 μm to 3.3 μm. In some embodiments, the thermal laser is a fiber laser configured to emit radiation having a wavelength in the range of 2.8 μm to 3.2 μm. In some embodiments, the thermal laser is a fiber laser configured to emit radiation having a wavelength in the range of 2.8 μm to 3.1 μm. In some embodiments, the thermal laser is a fiber laser configured to emit radiation having a wavelength in the range of 2.8 μm to 3.0 μm. In some embodiments, the thermal laser is a fiber laser configured to emit radiation having a wavelength in the range of 2.8 μm to 2.9 μm.

In some embodiments, the thermal laser is a fiber laser configured to emit radiation having a wavelength in the range of 2.9 μm to 3.4 μm. In some embodiments, the thermal laser is a fiber laser configured to emit radiation having a wavelength in the range of 3.0 μm to 3.3 μm. In some embodiments, the thermal laser is a fiber laser configured to emit radiation having a wavelength in the range of 3.1 μm to 3.2 μm.

In some embodiments, alternative lasers, which may or may not be thermal lasers, such as those targeting other target tissue chromophores, can also be used. For example, an Excimer laser in the wavelength range of 193 nm to 351 nm may be used in some embodiments of the present disclosure.

In some embodiments, the Excimer laser has a wavelength in the range of 193 nm to 350 nm. In some embodiments, the Excimer laser has a wavelength in the range of 193 nm to 325 nm. In some embodiments, the Excimer laser has a wavelength in the range of 193 nm to 300 nm. In some embodiments, the Excimer laser has a wavelength in the range of 193 nm to 275 nm. In some embodiments, the Excimer laser has a wavelength in the range of 193 nm to 250 nm. In some embodiments, the Excimer laser has a wavelength in the range of 193 nm to 225 nm. In some embodiments, the Excimer laser has a wavelength in the range of 193 nm to 200 nm.

In some embodiments, the Excimer laser has a wavelength in the range of 200 nm to 350 nm. In some embodiments, the Excimer laser has a wavelength in the range of 225 nm to 350 nm.

In some embodiments, the Excimer laser has a wavelength in the range of 250 nm to 350 nm. In some embodiments, the Excimer laser has a wavelength in the range of 300 nm to 350 nm. In some embodiments, the Excimer laser has a wavelength in the range of 325 nm to 350 nm.

In some embodiments, the Excimer laser has a wavelength in the range of 225 nm to 325 nm. In some embodiments, the Excimer laser has a wavelength in the range of 250 nm to 300 nm. In some embodiments, the Excimer laser has a wavelength of 275 nm.

In addition, a 355 nm triple-frequency neodymium-doped yttrium aluminum garnet (“Nd: YAG”) laser or a 266 nm fourth frequency Nd: YAG laser may be suitable for certain embodiments of the present disclosure.

The absorption coefficients and wavelengths of the Excimer and Nd: YAG lasers are also shown in FIG. 3 with their target chromophores shown in FIG. 2.

In some embodiments, the laser is configured to deliver radiation having a tissue absorption coefficient of 10 cm⁻¹ or more. In some embodiments, the tissue absorption coefficient can also range from 10 to 12,000 cm⁻¹, including all ranges therebetween. For example, in some embodiments, the tissue absorption coefficient ranges from 10 to 10,000 cm⁻¹. In some embodiments, the tissue absorption coefficient ranges from 10 to 5,000 cm⁻¹. In some embodiments, the tissue absorption coefficient ranges from 10 to 1,000 cm⁻¹. In some embodiments, the tissue absorption coefficient ranges from 10 to 500 cm⁻¹. In some embodiments, the tissue absorption coefficient ranges from 10 to 100 cm⁻¹. In some embodiments, the tissue absorption coefficient ranges from 10 to 50 cm⁻¹. In some embodiments, the tissue absorption coefficient ranges from 10 to 40 cm⁻¹. In some embodiments, the tissue absorption coefficient ranges from 10 to 30 cm⁻¹. In some embodiments, the tissue absorption coefficient ranges from 10 to 20 cm⁻¹.

In some embodiments, the tissue absorption coefficient ranges from 20 to 10,000 cm⁻¹. In some embodiments, the tissue absorption coefficient ranges from 50 to 10,000 cm⁻¹. In some embodiments, the tissue absorption coefficient ranges from 100 to 10,000 cm⁻¹. In some embodiments, the tissue absorption coefficient ranges from 500 to 10,000 cm⁻¹. In some embodiments, the tissue absorption coefficient ranges from 1,000 to 10,000 cm⁻¹. In some embodiments, the tissue absorption coefficient ranges from 5,000 to 10,000 cm⁻¹. In some embodiments, the tissue absorption coefficient ranges from 6,000 to 10,000 cm⁻¹. In some embodiments, the tissue absorption coefficient ranges from 7,000 to 10,000 cm⁻¹. In some embodiments, the tissue absorption coefficient ranges from 8,000 to 10,000 cm⁻¹. In some embodiments, the tissue absorption coefficient ranges from 9,000 to 10,000 cm⁻¹.

In some embodiments, the tissue absorption coefficient ranges from 20 to 5,000 cm⁻¹. In some embodiments, the tissue absorption coefficient ranges from 40 to 2500 cm⁻¹. In some embodiments, the tissue absorption coefficient ranges from 80 to 1200 cm⁻¹. In some embodiments, the tissue absorption coefficient ranges from 160 to 600 cm⁻¹. In some embodiments, the tissue absorption coefficient ranges from 300 to 320 cm⁻¹.

In some embodiments, the laser is configured to deliver radiation having a tissue absorption depth of below 0.6 mm.

In some embodiments, the laser is configured to deliver radiation having a tissue absorption depth ranging from 1 μm to 1 mm including all ranges therebetween. For example, in some embodiments, the laser is configured to deliver radiation having a tissue absorption depth ranging from 10 μm to 1 mm. In some embodiments, the laser is configured to deliver radiation having a tissue absorption depth ranging from 100 μm to 1 mm. In some embodiments, the laser is configured to deliver radiation having a tissue absorption depth ranging from 200 μm to 1 mm. In some embodiments, the laser is configured to deliver radiation having a tissue absorption depth ranging from 300 μm to 1 mm. In some embodiments, the laser is configured to deliver radiation having a tissue absorption depth ranging from 400 μm to 1 mm. In some embodiments, the laser is configured to deliver radiation having a tissue absorption depth ranging from 500 μm to 1 mm. In some embodiments, the laser is configured to deliver radiation having a tissue absorption depth ranging from 600 μm to 1 mm. In some embodiments, the laser is configured to deliver radiation having a tissue absorption depth ranging from 700 μm to 1 mm. In some embodiments, the laser is configured to deliver radiation having a tissue absorption depth ranging from 800 μm to 1 mm. In some embodiments, the laser is configured to deliver radiation having a tissue absorption depth ranging from 900 μm to 1 mm.

In some embodiments, the laser is configured to deliver radiation having a tissue absorption depth ranging from 100 μm to 900 μm. In some embodiments, the laser is configured to deliver radiation having a tissue absorption depth ranging from 100 μm to 800 μm. In some embodiments, the laser is configured to deliver radiation having a tissue absorption depth ranging from 100 μm to 700 μm. In some embodiments, the laser is configured to deliver radiation having a tissue absorption depth ranging from 100 μm to 600 μm. In some embodiments, the laser is configured to deliver radiation having a tissue absorption depth ranging from 100 μm to 500 μm. In some embodiments, the laser is configured to deliver radiation having a tissue absorption depth ranging from 100 μm to 400 μm. In some embodiments, the laser is configured to deliver radiation having a tissue absorption depth ranging from 100 μm to 300 μm. In some embodiments, the laser is configured to deliver radiation having a tissue absorption depth ranging from 100 μm to 200 μm.

In some embodiments, the laser is configured to deliver radiation having a tissue absorption depth ranging from 200 μm to 900 μm. In some embodiments, the laser is configured to deliver radiation having a tissue absorption depth ranging from 300 μm to 700 μm. In some embodiments, the laser is configured to deliver radiation having a tissue absorption depth ranging from 400 μm to 600 μm. In some embodiments, the laser is configured to deliver radiation having a tissue absorption depth of 500 μm.

In some embodiments, the laser is configured to deliver radiation having a wavelength of less than 11 μm. In some embodiments, the laser is configured to deliver radiation having a wavelength of less than 2 μm.

In some embodiments, the laser is configured to deliver radiation having a wavelength ranging from 1 nm to 11 μm including all ranges therebetween. For example, in some embodiments, the laser is configured to deliver radiation having a wavelength ranging from 2 nm to 11 μm. In some embodiments, the laser is configured to deliver radiation having a wavelength ranging from 5 nm to 11 μm. In some embodiments, the laser is configured to deliver radiation having a wavelength ranging from 10 nm to 11 μm. In some embodiments, the laser is configured to deliver radiation having a wavelength ranging from 50 nm to 11 μm. In some embodiments, the laser is configured to deliver radiation having a wavelength ranging from 100 nm to 11 μm. In some embodiments, the laser is configured to deliver radiation having a wavelength ranging from 250 nm to 11 μm. In some embodiments, the laser is configured to deliver radiation having a wavelength ranging from 500 nm to 11 μm. In some embodiments, the laser is configured to deliver radiation having a wavelength ranging from 1 μm to 11 μm. In some embodiments, the laser is configured to deliver radiation having a wavelength ranging from 2 μm to 11 μm. In some embodiments, the laser is configured to deliver radiation having a wavelength ranging from 5 μm to 11 μm. In some embodiments, the laser is configured to deliver radiation having a wavelength ranging from 5 μm to 10 μm.

In some embodiments, the laser is configured to deliver radiation having a wavelength ranging from 2 nm to 10 μm. In some embodiments, the laser is configured to deliver radiation having a wavelength ranging from 2 nm to 5 μm. In some embodiments, the laser is configured to deliver radiation having a wavelength ranging from 2 nm to 2 μm. In some embodiments, the laser is configured to deliver radiation having a wavelength ranging from 2 nm to 1 μm. In some embodiments, the laser is configured to deliver radiation having a wavelength ranging from 2 nm to 500 nm. In some embodiments, the laser is configured to deliver radiation having a wavelength ranging from 2 nm to 250 nm. In some embodiments, the laser is configured to deliver radiation having a wavelength ranging from 2 nm to 100 nm. In some embodiments, the laser is configured to deliver radiation having a wavelength ranging from 2 nm to 50 nm. In some embodiments, the laser is configured to deliver radiation having a wavelength ranging from 2 nm to 25 nm. In some embodiments, the laser is configured to deliver radiation having a wavelength ranging from 2 nm to 10 nm. In some embodiments, the laser is configured to deliver radiation having a wavelength ranging from 2 nm to 5 nm. In some embodiments, the laser is configured to deliver radiation having a wavelength ranging from 2 nm to 4 nm. In some embodiments, the laser is configured to deliver radiation having a wavelength ranging from 2 nm to 3 nm.

In some embodiments, the laser is configured to deliver radiation having a wavelength ranging from 2 nm to 5 μm. In some embodiments, the laser is configured to deliver radiation having a wavelength ranging from 10 nm to 1 μm. In some embodiments, the laser is configured to deliver radiation having a wavelength ranging from 50 nm to 500 nm. In some embodiments, the laser is configured to deliver radiation having a wavelength ranging from 100 nm to 200 nm. In some embodiments, the laser is configured to deliver radiation having a wavelength ranging from 150 nm to 175 nm.

In some embodiments, the laser can have any wavelength if the tissue absorption coefficient is above 10 cm⁻¹ or the absorption depth is below 0.6 mm.

In some embodiments, each pulse of the plurality of pulses 102 of laser radiation can have a duration ranging from 10 ns to 1 s. In some embodiments, each pulse of the plurality of pulses 102 of laser radiation can have a duration ranging from 50 ns to 1 s. In some embodiments, each pulse of the plurality of pulses 102 of laser radiation can have a duration ranging from 100 ns to 1 s. In some embodiments, each pulse of the plurality of pulses 102 of laser radiation can have a duration ranging from 500 ns to 1 s. In some embodiments, each pulse of the plurality of pulses 102 of laser radiation can have a duration ranging from to 1 s. In some embodiments, each pulse of the plurality of pulses 102 of laser radiation can have a duration ranging from 1 μs to 1 s. In some embodiments, each pulse of the plurality of pulses 102 of laser radiation can have a duration ranging from 5 μs to 1 s. In some embodiments, each pulse of the plurality of pulses 102 of laser radiation can have a duration ranging from 10 s to 1 s. In some embodiments, each pulse of the plurality of pulses 102 of laser radiation can have a duration ranging from 20 μs to 1 s. In some embodiments, each pulse of the plurality of pulses 102 of laser radiation can have a duration ranging from 50 μs to 1 s. In some embodiments, each pulse of the plurality of pulses 102 of laser radiation can have a duration ranging from 100 ms to 1 s. In some embodiments, each pulse of the plurality of pulses 102 of laser radiation can have a duration ranging from 1 ms to 1 s. In some embodiments, each pulse of the plurality of pulses 102 of laser radiation can have a duration ranging from 10 ms to 1 s. In some embodiments, each pulse of the plurality of pulses 102 of laser radiation can have a duration ranging from 100 ms to 1 s. In some embodiments, each pulse of the plurality of pulses 102 of laser radiation can have a duration ranging from 200 ms to 1 s. In some embodiments, each pulse of the plurality of pulses 102 of laser radiation can have a duration ranging from 300 ms to 1 s. In some embodiments, each pulse of the plurality of pulses 102 of laser radiation can have a duration ranging from 400 ms to 1 s. In some embodiments, each pulse of the plurality of pulses 102 of laser radiation can have a duration ranging from 500 ms to 1 s. In some embodiments, each pulse of the plurality of pulses 102 of laser radiation can have a duration ranging from 600 ms to 1 s. In some embodiments, each pulse of the plurality of pulses 102 of laser radiation can have a duration ranging from 700 ms to 1 s. In some embodiments, each pulse of the plurality of pulses 102 of laser radiation can have a duration ranging from 800 ms to 1 s. In some embodiments, each pulse of the plurality of pulses 102 of laser radiation can have a duration ranging from 900 ms to 1 s.

In some embodiments, each pulse of the plurality of pulses 102 of laser radiation can have a duration ranging from 10 ns to 500 ms. In some embodiments, each pulse of the plurality of pulses 102 of laser radiation can have a duration ranging from 10 ns to 100 ms. In some embodiments, each pulse of the plurality of pulses 102 of laser radiation can have a duration ranging from 10 ns to 10 ms. In some embodiments, each pulse of the plurality of pulses 102 of laser radiation can have a duration ranging from 10 ns to 1 ms. In some embodiments, each pulse of the plurality of pulses 102 of laser radiation can have a duration ranging from 10 ns to 100 μs. In some embodiments, each pulse of the plurality of pulses 102 of laser radiation can have a duration ranging from 10 ns to 50 μs. In some embodiments, each pulse of the plurality of pulses 102 of laser radiation can have a duration ranging from 10 ns to 40 μs. In some embodiments, each pulse of the plurality of pulses 102 of laser radiation can have a duration ranging from 10 ns to 30 μs. In some embodiments, each pulse of the plurality of pulses 102 of laser radiation can have a duration ranging from 10 ns to 20 μs. In some embodiments, each pulse of the plurality of pulses 102 of laser radiation can have a duration ranging from 10 ns to 10 ns. In some embodiments, each pulse of the plurality of pulses 102 of laser radiation can have a duration ranging from 10 ns to 5 μs. In some embodiments, each pulse of the plurality of pulses 102 of laser radiation can have a duration ranging from 10 ns to 1 μs. In some embodiments, each pulse of the plurality of pulses 102 of laser radiation can have a duration ranging from 10 ns to 100 ns. In some embodiments, each pulse of the plurality of pulses 102 of laser radiation can have a duration ranging from 10 ns to 50 ns.

In some embodiments, each pulse of the plurality of pulses 102 of laser radiation can have a duration ranging from 10 ns to 100 ms. In some embodiments, each pulse of the plurality of pulses 102 of laser radiation can have a duration ranging from 100 ns to 10 ms. In some embodiments, each pulse of the plurality of pulses 102 of laser radiation can have a duration ranging from 1 μs to 10 ms. In some embodiments, each pulse of the plurality of pulses 102 of laser radiation can have a duration ranging from 10 μs to 1 ms. In some embodiments, each pulse of the plurality of pulses 102 of laser radiation can have a duration ranging from 50 μs to 500 μs. In some embodiments, each pulse of the plurality of pulses 102 of laser radiation can have a duration of 100 μs.

The duration, frequency and fluence of the pulses can be varied by a skilled artisan so long as those variations cause minimal trauma to the target tissue the tissue while still ablating the tissue.

In some embodiments, the fiber optic probe 101 is inserted into the eye through a corneal incision.

In some embodiments, the fiber optic probe 101 is inserted into the eye by perforating of the distal end 101a fiber optic probe 101 and penetrating the perforated end directly into the eye.

In some embodiments, the fiber optic probe 101 is guided for placement in contact with or adjacent to the target tissue (i.e. trabecular meshwork, Schwalbe's line, or any point in the range between the scleral spur and the sclerocorneal junction) through microscopic observation. The microscopic observation can be aided by an aiming beam, which can radiate from the distal end 101a for the fiber optic probe. In some embodiments, the aiming beam is on the visible spectrum. In some embodiments, the aiming beam can also be used as an accessory tool for further guidance.

In some embodiments, the fiber optic probe is guided for placement in contact with or adjacent to the target tissue (i.e. trabecular meshwork, Schwalbe's line, or any point in the range between the scleral spur and the sclerocorneal junction) by a goniolens.

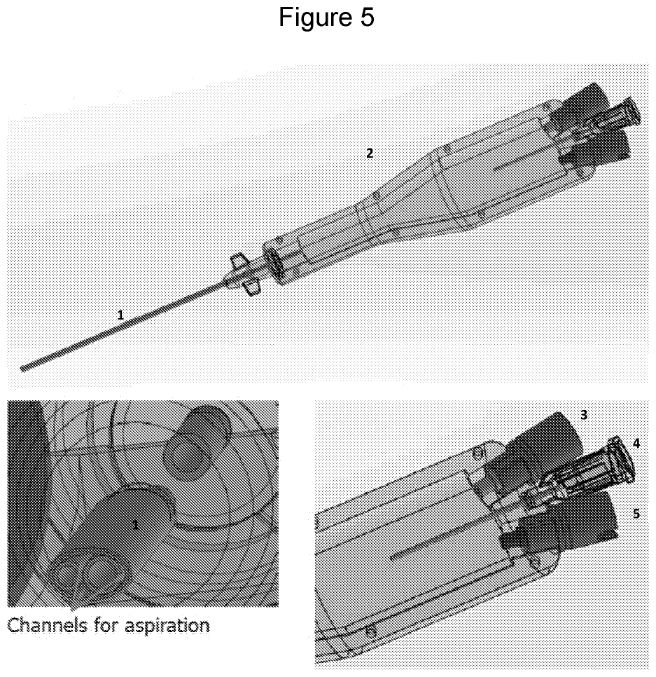

In some embodiments, the fiber optic probe 101 is guided for placement adjacent to or in contact with the target tissue (i.e. trabecular meshwork, Schwalbe's line, or any point in the range between the scleral spur and the sclerocorneal junction) by coupling the fiber optic probe 101a with an endoscope. The endoscope can comprise a camera and a light (e.g., as in FIG. 5). In some embodiments, the endoscope has an aspiration mechanism for better control and guidance of the probe to the target tissue that was defined as the entrance point for the fiber optic.

In some embodiments, the fiber probe is bent with a bending radius of up to 40°. In some embodiments, the bending can enable better control and maneuvering of the fiber optic inside the eye.

In some embodiments, the material of the fiber optic probe 101 can comprise at least one of a solid core fiber or a hollow core waveguide (HCW). The fiber optic probe 101 can further comprise one or more fiber tips and a solid core fiber inserted into a protecting medical grade tube, such as a stainless-steel, nitinol or titanium tube. This serves to increase hardness and rigidity of the fiber optic probe 101 and prevent direct heat dispersion to adjacent tissues. The HCW can comprise an optical window at an exit portion. This optical window can comprise at least one of a diamond or zinc-selenium (“Zn: Se”) material. In some embodiments, the fiber optic probe can be connected to a handpiece. The HCW can also prevent liquid from getting into one or more fiber tips of the fiber optic probe 101.

In some embodiments of the present disclosure, the method and device can be included as part of an irrigation-aspiration system. The air irrigation-aspiration system can have several functionalities including enabling laser transmission for highly absorbed lasers by water. For example, in some embodiments where, the laser wavelength is highly absorbed by water-based material, some of the pulses 102 of laser radiation may not be effective on the liquid environment inside of the eye. Therefore, if the distal end of the fiber is not equipped with a protecting window (e.g. diamond or ZnSe), for the laser to be effective, the medium for the laser delivery may need to be changed to air. Accordingly, in some embodiments, the air irrigation-aspiration system is configured to inject air bubbles synchronized with the emission of the plurality of pulses 102 of later radiation.

In some embodiments, to prevent high air pressure inside the eye, in order not to generate high pressure by the air bubbles, and to improve the coupling of the probe to the target tissue, the aspiration of the air should be applied in parallel to the air injection and laser emission.

In some embodiments, the irrigation aspiration system also serves as a cooling system, which allows heat generated by the laser transmission through a portion of the fiber to be drawn back in to a portion of the fiber. In some embodiments, this can occur through the drawing in of heated air back into the fiber optic probe 101.

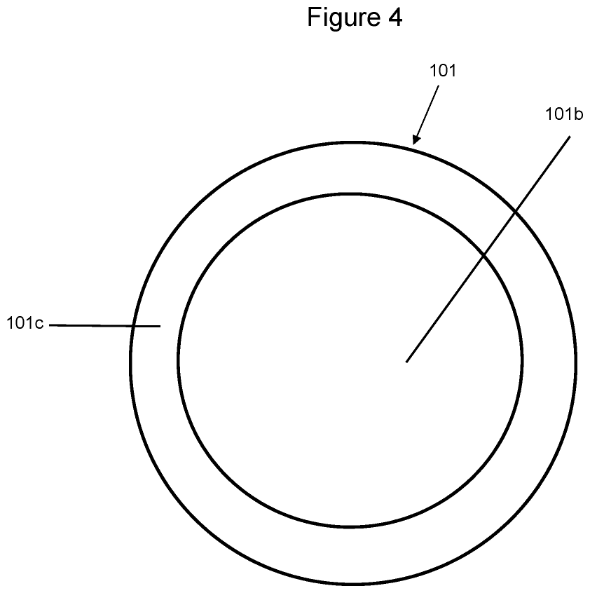

In some embodiments, such as the embodiment of FIG. 4, the fiber optic probe 101 can comprise an inner annulus 101b and an outer annulus 101c. In some embodiments, the inner annulus 101b releases a fluid, such as air, into the eye, thereby irrigating the eye. In some embodiments, the irrigation fluid can have a temperature T₁. In some embodiments, the outer annulus 101c is configured to aspirate or draw heated fluid from the eye back in to the fiber optic probe 101. In some embodiments, the outer annulus 101c of the fiber optic probe 101 has a temperature T₂. As can be understood by those skilled in the art, because the plurality of pulses 102 of laser radiation heat the irrigation fluid, T₂ can be greater than T₁, such that the aspiration of fluid into the outer annulus cools the eye. In some embodiments, the inner annulus 101b also transmits the plurality of pulses 102 of laser radiation.

In some embodiments, viscoelastic material is injected into the anterior chamber. In some embodiments, an anterior chamber maintainer can also be used in addition to, or in conjunction with the viscoelastic material. In some embodiments, a bleb can form after the removal of viscoelastic material from the anterior chamber.

In some embodiments, a liquid material, such as an anti-fibrotic material is injected into the subconjunctival space. In some embodiments, the injection may occur pre-operation or prior to using of the laser device. This anti-fibrotic material can comprise one or more of mitomycin-C (“MMC”) or fluorouracil (“5-FU”). The subconjunctival liquid material can absorb energy transmitted through all thickness of the sclera tissue before reaching the conjunctiva, which can prevent damage to the conjunctiva.

In some embodiments, at least one of topical, peribulbar, or retrobulbar local anesthesia is used.

In some embodiments the location for the fiber optic probe 101 in can be marked with a tissue marker prior to insertion. In some embodiments an exit from the sclera can be located 3 mm anterior to the limbus.

In some embodiments, the corneal entry point of the fiber optic probe is at least 1 to 2 mm anterior to the limbus, which can enable probe exit positioning in the sclera 2-6 mm anterior from the limbus.

Once the fiber optic probe is aligned with the desired entry point in the anterior chamber angle, the surgeon should start operating the laser (i.e. perform laser ablating) and advance the laser fiber in the anterior chamber angle and sclera until the surgeon is able to visualize the fiber-tip as it exits the sclera into the subconjunctival space. In some embodiments, an area for fiber-tip exit at subconjunctival space should be 2-6 mm anterior to the limbus. In some embodiments, an area for fiber-tip exit at subconjunctival space should be 2-5 mm anterior to the limbus. In some embodiments, an area for fiber-tip exit at subconjunctival space should be 2-4 mm anterior to the limbus. In some embodiments, an area for fiber-tip exit at subconjunctival space should be 2-3 mm anterior to the limbus.

In some embodiments, an area for fiber-tip exit at subconjunctival space should be 3-6 mm anterior to the limbus. In some embodiments, an area for fiber-tip exit at subconjunctival space should be 4-6 mm anterior to the limbus. In some embodiments, an area for fiber-tip exit at subconjunctival space should be 5-6 mm anterior to the limbus.

In some embodiments, the intended area can be marked with a tissue marker prior to probe insertion.

In some embodiments, the fiber optic probe inserted into the patient's eye has a dimeter ranging from 50 μm to 300 μm including all ranges therebetween. For instance, in some embodiments, the fiber optic probe inserted into the patient's eye is has a dimeter ranging from 100 μm to 300 μm. In some embodiments, the fiber optic probe inserted into the patient's eye is has a dimeter ranging from 150 μm to 300 μm. In some embodiments, the fiber optic probe inserted into the patient's eye is has a dimeter ranging from 200 μm to 300 μm. In some embodiments, the fiber optic probe inserted into the patient's eye is has a dimeter ranging from 250 μm to 300 μm. In some embodiments, the fiber optic probe inserted into the patient's eye is has a dimeter ranging from 50 μm to 250 μm. In some embodiments, the fiber optic probe inserted into the patient's eye is has a dimeter ranging from 100 μm to 250 μm. In some embodiments, the fiber optic probe inserted into the patient's eye is has a dimeter ranging from 150 μm to 250 μm. In some embodiments, the fiber optic probe inserted into the patient's eye is has a dimeter ranging from 200 μm to 250 μm.

In some embodiments, the fiber optic probe inserted into the patient's eye is has a dimeter ranging from 50 μm to 200 μm. In some embodiments, the fiber optic probe inserted into the patient's eye is has a dimeter ranging from 100 μm to 150 μm.

A further non-limiting embodiment of a fiber optic probe according to the present disclosure is shown in FIG. 5. As shown, the fiber optic probe may include a disposable part 1. The disposable part 1 may include an optical fiber (not shown) and an imaging probe (not shown). The fiber optic probe may further include a handpiece 2. The handpiece 2 may include connectivity to several different modules. The fiber optic probe may also include at least one of: a laser connectivity port 3, an aspiration connectivity port 4, an imaging and illumination connectivity port 5, or any combination thereof.

While several embodiments of the present disclosure have been described, it is understood that these embodiments are illustrative only, and not restrictive, and that many modifications may become apparent to those of ordinary skill in the art. For example, all dimensions discussed herein are provided as examples only, and are intended to be illustrative and not restrictive.

1. A method comprising:
obtaining a fiber optic probe,
wherein the fiber optic probe comprises a distal end;
introducing the fiber optic probe between an outer surface of an eye and an anterior chamber of an eye;
advancing the fiber optic probe across the anterior chamber of the eye so that the fiber optic probe is adjacent to or in contact with: the trabecular meshwork, Schwalbe's line, between the scleral spur and the sclerocorneal junction, or any combination thereof;
delivering a plurality of pulses of laser radiation through a laser and into the eye;
wherein the laser is disposed at a distal end of the fiber optic probe;
ablating ocular tissue of the eye with the plurality of pulses of laser radiation,
wherein the ablating generates a drainage channel, and
wherein the drainage channel extends from the anterior chamber of the eye to the subconjunctival space of the eye.

2. The method of claim 1, wherein the ablating is thermal ablating and the laser radiation is thermal laser radiation.

3. The method of claim 1, wherein the fiber optic probe is inserted into the eye directly through perforation by the fiber optic probe, through a corneal incision, or any combination thereof.

4. The method of claim 1, wherein the fiber optic probe is guided for placement in contact with or adjacent to the trabecular meshwork, Schwalbe's line, between the scleral spur and the sclerocorneal junction, or any combination thereof through microscopic observation.

5. The method of claim 1 wherein, the fiber optic probe is guided for placement in contact with or adj acent to the trabecular meshwork, Schwalbe's line, between the scleral spur and the sclerocorneal junction, or any combination thereof by a goniolens.

6. The method of claim 1, wherein the fiber optic probe is guided for placement adjacent to or in contact with the the trabecular meshwork, Schwalbe's line, between the scleral spur and the sclerocorneal junction, or any combination thereof by coupling the fiber optic probe with an endoscope.

7. The method of claim 1, wherein the fiber optic probe has a diameter ranging from 50 μm to 300 μm.

8. The method of claim 1, wherein the laser is configured to deliver radiation having a tissue absorption depth ranging from 1 μm to 0.6 mm.

9. The method of claim 1, wherein the laser is configured to deliver radiation having a tissue absorption coefficient ranging from 10 cm⁻¹ to 12,000 cm⁻¹.

10. The method of claim 1, wherein the laser is configured to deliver radiation having a wavelength ranging from 1 nm to 11 μm.

11. The method of claim 1, wherein the laser comprises one or more of: an EerbiumChromium doped Yttrium Scandium Gallium Garnet laser, a fiber laser, a quantum cascade laser, a Holmium doped Yttrium Scandium Gallium Garnet laser, or a fiber laser.

12. The method of claim 1, wherein the laser is a carbon dioxide laser.

13. The method of claim 1, wherein the laser is a fiber laser configured to emit radiation having a wavelength in the range of 2.8 μm to 3.5 μm.

14. The method of claim 1, wherein each pulse of the plurality of pulses of laser radiation has a duration ranging from 10 ns to 1 s.

15. The method of claim 1, wherein the fiber optic probe comprises a solid core fiber.

16. The method of claim 1 wherein, the fiber optic probe comprises a hollow core waveguide.

17. The method of claim 1, wherein the fiber optic probe comprises an inner annulus and an outer annulus, the method further comprising the steps of:
emitting a fluid from the inner annulus of the fiber optic probe thereby irrigating the eye, the fluid having a temperature T₁; and
aspirating fluid from the eye into the outer annulus of the fiber optic probe the air having a temperature T₂; wherein T₂>T₁, such that the receipt of fluid into the outer annulus cools the eye.

18. The method of claim 1, further comprising injecting a liquid or viscoelastic material into the subconjunctival space of the eye, wherein the liquid material comprises at least one anti-fibrotic material.

19. The method of claim 1 further comprising step of injecting viscoelastic material into the anterior chamber of the eye.

20. The method of claim 1, wherein the fiber optic probe is straight.

21. The method of claim 1, wherein the fiber optic probe is bent.

22. The method of claim 1, wherein the fiber optic probe is adjacent to or in contact with the trabecular meshwork, Schwalbe's line, between the scleral spur and the sclerocorneal junction, or any combination thereof from within the anterior chamber.

23. The method of claim 1, wherein the step of advancing the fiber optic probe comprises advancing the fiber optic probe automatically or manually in a manner correlated with a rate of ocular tissue ablation and drainage channel generation by the pulses of laser radiation, thereby maintaining the distal end of the fiber optic probe in contact with the ocular tissue.
